(19) United States
(12) Patent Application Publication
(10) Pub. No.: US 2008/0065196 A1
(43) Pub. Date: Mar. 13, 2008

(54) Intra-Columnar Cell Features to Improve Drug Distribution and Scaffolding of a Stent

(76) Inventors: Michael Wayne Davis, Rockford, MN (US); Brian J. Brown, Hanover, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/844,583

(22) Filed: Aug. 24, 2007

Related U.S. Application Data
(60) Provisional application No. 60/843,873, filed on Sep. 12, 2006.

(51) Int. Cl. A61F 2/82

(52) U.S. Cl. 623/1.16; 623/1.41; 623/1.42

(57) ABSTRACT

The present invention is directed toward a stent having a plurality of axially spaced serpentine bands, each serpentine band having an axis circumferentially oriented around the longitudinal axis of the stent. The serpentine bands have a plurality of struts spaced along the axis of the serpentine band forming alternating peaks and troughs. The serpentine bands are interconnected via a plurality of interconnecting struts to form a plurality of cells defined by axially adjacent serpentine bands and circumferentially adjacent interconnecting struts. When the stent is in its unexpanded state, each serpentine comprises a plurality of slits, each slit being non-linear and continuous from a first end to a second end and being formed in at least a portion of each of three consecutively connected struts. Upon expansion of the stent to its expanded state, each of the slits expands in size to form an intra-columnar cell (ICC).

### CROSS-REFERENCE TO RELATED APPLICATIONS

This application claims the benefit of U.S. Provisional Application No. 60/843,873, filed on Sep. 12, 2006, the entire content of which is hereby incorporated by reference.

### FIELD OF THE INVENTION

This invention relates to implantable medical devices, such as stents, their manufacture, delivery and methods of use.

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable). An example of a balloon expandable stent is shown in U.S. Pat. No. 5,843,120. An example of a self-expanding stent is described in WO 96/26689.

Stents may be created by methods including cutting or etching a design from a tubular stock, from a flat sheet which is cut or etched and which is subsequently tolled or from one or more interwoven wires or braids.

There remains a need for stent patterns that provide proper scaffolding support and drug delivery in the expanded state, while also allowing for crimpability and for flexibility and deliverability in the unexpanded state.

The art referred to and/or described above is not intended to constitute an admission that any patent, publication or other information referred to herein is “prior art” with respect to this invention. In addition, this section should not be construed to mean that a search has been made or that no other pertinent information as defined in 37 C. F. R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

The present invention is directed toward a stent having a plurality of axially spaced serpentine bands, each serpentine band having an axis circumferentially oriented around the longitudinal axis of the stent. The serpentine bands have a plurality of struts spaced along the axis of the serpentine band forming alternating peaks and troughs. The serpentine bands are interconnected via a plurality of interconnecting struts to form a plurality of cells defined by axially adjacent serpentine bands and circumferentially adjacent interconnecting struts. When the stent is in its unexpanded state, each serpentine band comprises a plurality of slits within the serpentine band, each slit being non-linear and continuous from a first end to a second end. Upon expansion of the stent to its expanded state, each of the slits expands in size to form an intra-columnar cell (ICC). In some embodiments of the invention, each slit, from its first end to its second end, is formed in at least a portion of each of three consecutively connected struts.

In some embodiments, the slits are formed in three consecutively connected struts in a serpentine band. The three connected struts include a first strut, a second strut and third strut. The first, second and third struts each have a first end and a second end. The second end of the first strut is connected to the first end of the second strut and the second end of the second strut is connected to the first end of the third strut. The first end of the slit is positioned in the first strut and the second end of the slit is positioned in the third strut.

In some embodiments, the slit in the three consecutive struts crosses the axis of the serpentine band at least two times. In some embodiments, the slit crosses the axis of the serpentine band three times.

In some embodiments, the slits are formed in three consecutively connected struts in a serpentine band. The three connected struts include a first strut, a second strut and third strut. Each of the first, second and third struts have a first segment and a second segment, wherein the slit separates the first segment and second segment of the each of the first, second and third struts.

In some embodiments, the first segment of the first strut is connected to the first segment of the second strut forming a trough, the first segment of the second strut is connected to the first segment of the third strut forming a peak, the second segment of the first strut is connected to the second segment of the second strut forming a peak and the second segment of the second strut is connected to the second segment of the third strut forming a trough.

In some embodiments of the invention, upon expansion of the stent to its expanded state, each of the slits expands in size to form an intra-columnar cell (ICC). The ICC is substantially a polygon and has at least two inner reflex angles. In some embodiments, the ICC has at least two inner acute angles. In some embodiments, the ICC has at least four inner angles less than 180°.

In some embodiments, the first and second segments of the first, second and third struts define the ICC and the first segment of the first strut is substantially parallel with the second segment of the third strut. In some embodiments, the second segment of the first strut is substantially parallel with the first segment of the third strut. In some embodiments, the first segment of the second strut is substantially parallel with the second segment of the second strut.

In some embodiments, the second segment of the first strut and the first segment of the third strut have greater widths than the first segment of the first strut and the second segment of the third strut. In some embodiments, the first segment and second segment of the second strut may vary in their width along their lengths.

The serpentine bands may also comprise a plurality of primary hinge points, wherein, upon expansion of the stent and the forming of the ICC, the first and second segments of the first, second and third struts rotate around the primary hinge points, increasing the size of the slit to form the ICC. In some embodiments, a first primary hinge point is located in an end of the first segment of the second strut, wherein the first segment of the second strut and the first segment of the third strut pivot around the first primary hinge point A second primary hinge point may be located in an end of the second segment of the second strut, wherein the second segment of the second strut and the second segment of the first strut pivot around the second primary hinge point. There also may be six primary hinge points.

In some embodiments, the at least a portion of the stent is configured to include one or more mechanisms for the delivery of a therapeutic agent. Often the agent will be in the form of a coating or other layer (or layers) of material placed on a surface region of the stent and is adapted to be released at the site of the stent's implantation or areas adjacent thereto. The therapeutic and/or polymeric coatings may comprise one or more non-genetic therapeutic agents, genetic materials and cells and combinations thereof.

In some embodiments, an embodiment of the inventive stent is mounted on a stent delivery catheter. The present invention also further includes methods of delivering the disclosed inventive stents to a target site in a bodily vessel.

These and other embodiments that characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described an embodiments of the invention.

### BRIEF DESCRIPTION OF THE DRAWINGS

A detailed description of the invention is hereafter described with specific reference being made to the drawings.

FIG. 1 is a perspective view of an embodiment of the invention.

FIG. 2 is an expanded view of a portion 2 of the embodiment of FIG. 1.

FIG. 2A is an expanded view of a portion 2 of the embodiment of FIG. 1.

FIG. 3 is an expanded view of a portion 2 of the embodiment of FIG. 1 in its expanded state.

FIG. 4 is an expanded view of a portion of the embodiment of FIG. 1.

FIG. 5 is a partial view illustrating an embodiment of the invention.

FIG. 6 is a partial view illustrating an embodiment of the invention.

FIG. 7 is a partial view illustrating an embodiment of the invention.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

Depicted in the figures are various aspects of the invention. Elements depicted in one figure may be combined with, or substituted for, elements depicted in another figure as desired.

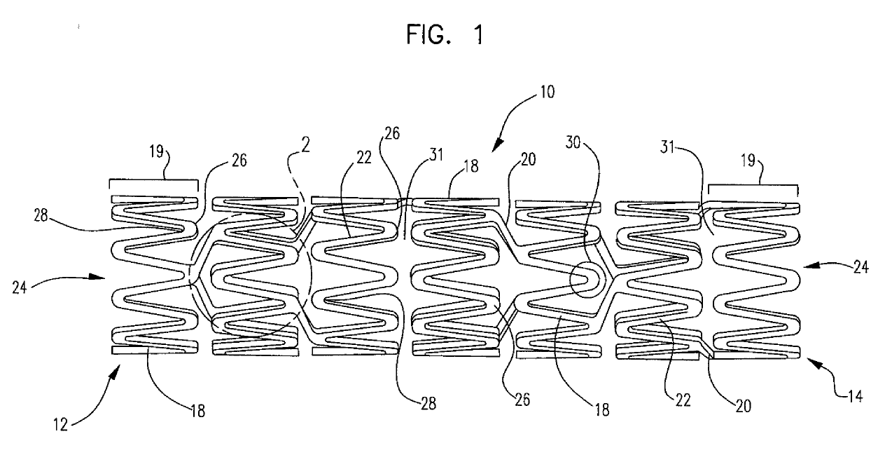

In one embodiment, as shown in FIG. 1, the invention is directed to a comprising a plurality of axially spaced serpentine bands 18. Each serpentine band 18 is shaped in a tubular form forming a plurality of expansion columns 19. Each serpentine band 18, and therefore each expansion column 19, is connected to a longitudinally adjacent serpentine band 18 via a plurality of interconnecting struts 20. The interconnecting struts shown are of substantially the same length and arranged in a uniform patter. However, it should be understood that the interconnecting struts 20 may vary in design, numbered length and pattern.

A plurality of cells 31 are defined by longitudinally adjacent serpentine bands 18 and circumferentially adjacent interconnecting struts 20. It should be understood that the shape of the cells vary and the cell pattern may be uniform or irregular.

The serpentine bands 18 comprise struts 22 circumferentially arranged around the longitudinal axis 24 of the stent 10. Adjacent struts 22 are connected to one another forming alternating peaks 26 and troughs 28. Since the junctures 30 between adjacent struts 22 could be considered to form a peak 26 and a trough 28 from a top view, the alternating peaks 26 and troughs 28 characteristic should be considered from a proximal end to a distal end or a distal end to a proximal end perspective. It should be understood that the present invention contemplates other generally serpentine configurations and not just the exact configuration shown.

The stent 10 has a contracted condition, as shown in FIG. 1, and an expanded condition. When the stent 10 is expanded, the diameters of the expansion columns 19 increase, the distance between circumferentially adjacent peaks 26 increases and the troughs become more obtuse.

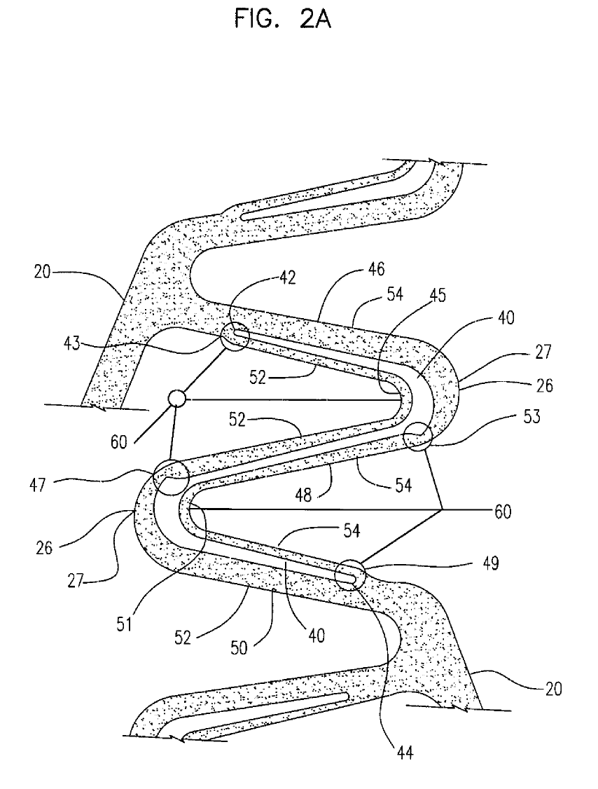

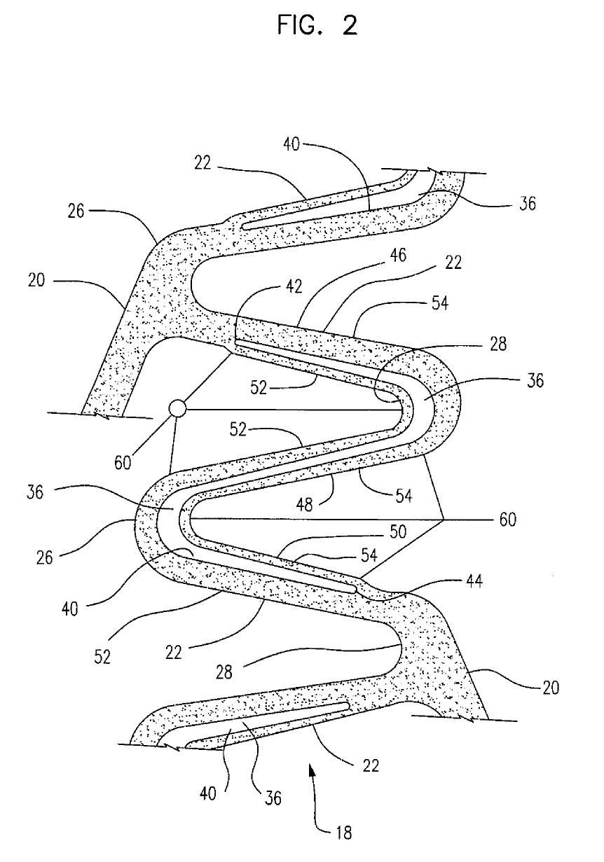

FIG. 2 is an expanded view of a portion 2 of the stent of FIG. 1. As can be seen from this illustration of a portion of a non-expanded serpentine band 18, the serpentine bands 18 further include intra-columnar cells (ICC) 36. The ICC's 36 are intra-columnar in that they are within the serpentine band 18 as opposed to between the bands 18, as with cells 31. The ICC's 36 are visible in FIG. 2, however it should be understood that, when the stent 10 is in its unexpanded state, as shown in FIG. 2, the framework that defines and ICC 36 is contracted to such an extent that the ICC's 36 constitute slits 40 comprising very little or no open space. These slits 40 are long narrow non-linear cuts or openings fully through the serpentine bands 18 separating adjacent segments 52/54 of a strut 22. As mentioned above, the slits 40 may comprise a visual opening, but may be closed down to such an extent that the segments 52/54 of a particular strut 22 that partially define a slit 40 touch one another. They may be formed by cutting, for example laser cutting, or by any other suitable manner.

As shown in FIG. 2, when the stent 10 is in its contracted state, the ICC's 36 are slits 40. The slits 40 have a first end 42 continuously extending to a second end 44. In the embodiment shown in FIG. 2, within a single serpentine band 18, the first end 42 of the slit 40 starts in a first strut 46, continues in an adjacent second strut 48 and terminates at the second end 44 in a consecutive third strut 50. The slits 40 take on a backward or forward “S” shape as viewed when the stent 10 is horizontal, as shown in FIG. 11n some embodiments, the pattern along the serpentine band is an alternating pattern of backward and forward “S” shapes.

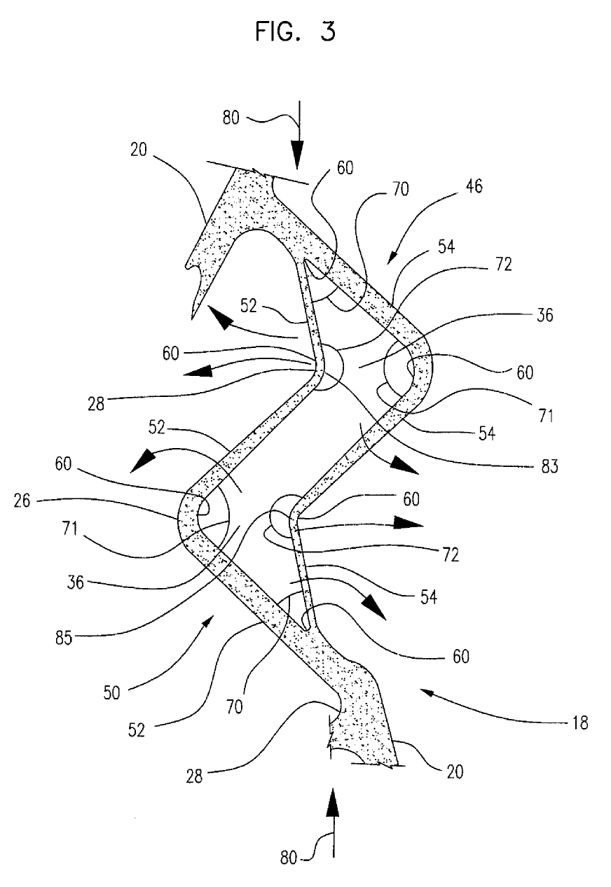

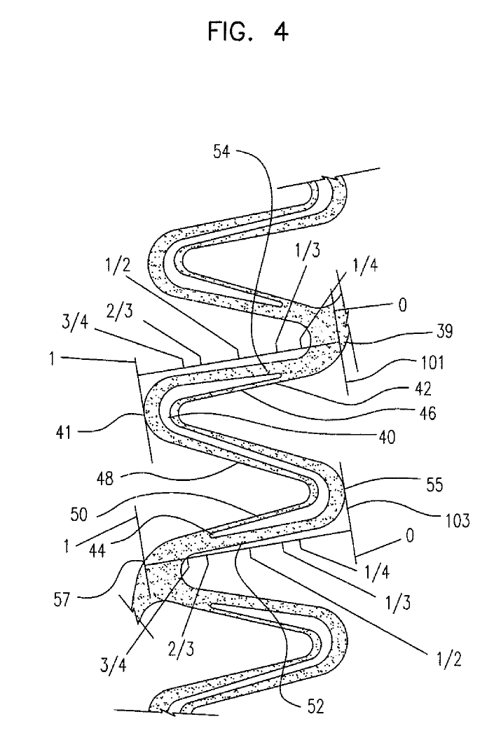

FIG. 4 shows a portion of a serpentine band 18 with first strut 46, second strut 48 and third strut 50. The slit 40 shown in FIG. 4 is a forward “S” shape as opposed to the backward “S” shape shown in FIG. 2-3. The description is the same, differing only in that it is a mirror image. The slit 40 extends from its first end 42 in strut 46 to its second end 44 in strut 50. A ruler 101 is shown extending from a first end 39 of strut 46 to a second end 41. The ruler 101 is position parallel with segment 54 of the strut 46. A ruler 103 is also shown extending from a first end 55 of strut 50 to a second end 57. The ruler 103 is position parallel with segment 52 of the strut 50.

Both rulers 101, 103, are squared off at the ends 39, 41, 55, 57, of the measured struts 46, 50. The rulers 101, 103, start at 0 and go to 1, which indicates the complete length. The intermediate hashes are proportional measurements. For example, the length of strut between 0 and ½ indicates the first or beginning half of the strut and, similarly, the length of strut between ½ and 1 indicates the second or last half of the strut.

In the embodiment shown, using the rulers 101, 103, the slit 40 starts 42 in the first ⅓ of strut 46 and ends 44 in the last ⅓ of strut 50. Specifically, the start 42 point is between about ¼ and about ⅓ of strut 46 and the end 44 is between about ⅔ and about ¼ of strut 50. In some embodiments, the slit 40 may start 42 in the first ¼ (between and including 0 and ¼) of strut 46 and may end 44 in the last ¼ (between and including ¾ and 1) of strut 50. In some embodiments, the slit 40 may start 42 in the first ⅓ (between and including 0 and ⅓) of strut 46 and may end 44 in the last ⅓ (between and including ⅔ and 1) of strut 50. In some embodiments, the slit 40 may start 42 in the first ½ (between and including 0 and ½) of strut 46 and may end 44 in the may start 42 in the first ⅔ (between and including 0 and ⅔) of strut 46 and may end 44 in the last ⅔ (between and including ⅓ and 1) of strut 50. In some embodiments, the slit 40 may start 42 between the first ¼ and final ¼ (between and including ¼ and ¾) of strut 46 and may end 44 between the first ¼ and final ¼ (between and including ¼ and ¾) of strut 50.

The present invention also contemplates different combinations of the starting 42 and ending 44 points mentioned above. For example, in some embodiments, the slit 40 may start in the first ⅓ (between and including 0 and ⅓) of strut 46 and may end 44 in the last ½ (between and including ½ and 1) of strut 50, etc. The invention also contemplates starting points 42 and ending points 44 between and including the specific individual hash marks shown on the rulers 101, 103. For example, between and including 1 and ⅓; between and including ½ and ⅔; between and including ⅓ and ⅔, etc. The size and opening properties of the ICC 36 can be controlled by adjusting the locations of the starting point 42 and ending point 44 of the slit 40.

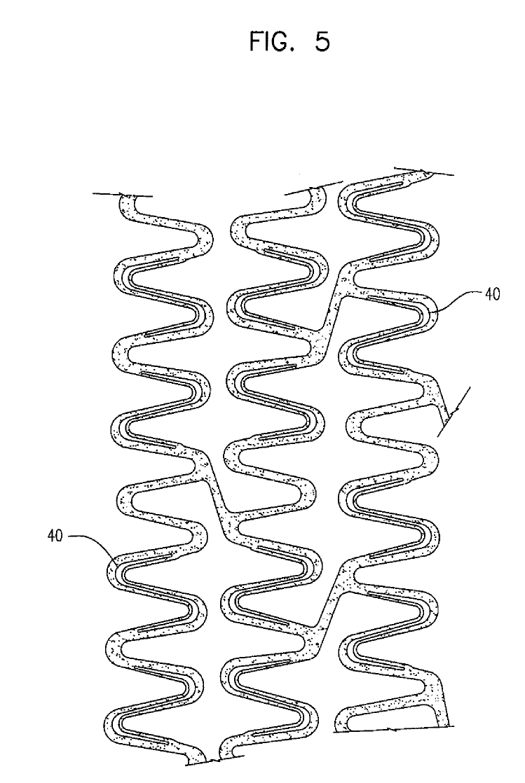

The slit 40, shown in FIG. 2, splits the first 46, second 48 and third 50 struts into respective first 52 and second 54 segments. When the stent 10 is in its non-expanded form, as shown in FIG. 2, each first segment 52 is immediately adjacent to its corresponding second segment 54. Each serpentine band 18 may contain a plurality of slits 40. In some embodiments, the slits 40 may be uniformly arranged and, in some embodiments, the slits 40 may be arranged in a non-uniform manner A non-limiting example of a pattern of slits 40 of an embodiment of the invention is shown in FIG. 5. It should be understood that the pattern may vary.

FIG. 2A is identical to FIG. 2 except that it is slightly enlarged for further illustration. In this FIG. 2A, primary hinge points 60 are shown in the serpentine band 18 along the slit 40. In the embodiment shown, there are six primary hinge points (PHP). As can be seen in FIG. 2A, a first PHP 43 is located at the end of segment 52 of strut 46 at the first end 42 of the slit 40. A second PHP 45 is located in the trough 28 between segment 52 of strut 46 and segment 52 of strut 48. A third PHP 47 is located in the end of segment 52 of strut 48 adjacent to the peak 26 between segment 52 of strut 48 and segment 52 of strut 50 PHP 47 is offset from the apex 27 of the peak 26 on the side of segment 52 of strut 48. A fourth PHP 49 is located at the end of segment 54 of strut 50 at the second end 44 of the slit 40. A fifth PHP 51 is located in the trough 28 between segment 54 of strut 48 and segment 54 of strut 50. And a sixth PHP 53 is located in the end of segment 54 of strut 48 adjacent to the peak 26 between segment 54 of strut 48 and segment 54 of strut 46 PHP 53 is offset from the apex 27 of the peak 26 on the side of segment 54 of strut 48. Upon expansion of the stent 10, the segments (52, 54) of the struts (46, 48, 50) associated with the slit 40/ICC 36, rotate around the primary hinge points 60.

The PHP may be thinned spots where plastic deformation may occur upon expansion. Positioning and design of the hinge points may also be designed using Finite Element Analysis to create stress risers that are not readily noticeable to dictate the PHP. The hinge points are located at or near the vertices of the polygon ICC structure. As the ICC structure is enlarged, the vertices of the ICC structure hinge and deform to allow the polygon shape to enlarge.

Upon radial expansion of the stent 10, as seen in FIG. 3, the ICCs 36 increase in area from a slit 40 to a full ICC 36. The arrows in FIG. 3 show the direction of movement of the segments 52, 54, of the struts 46, 48, 50, that define the ICC 36. The lengthening of the serpentine bands 18 in a circumferential direction causes the rotation of the segments 52, 54, of the struts 46, 48, 50, around the primary hinge points 60.

Upon expansion, the resulting ICC 36 shape is polygonal. The specific inner angle sizes and arrangements may vary upon deployment. It will open up as needed per the final deployed diameter A stent deployed to 5 mm may have greater angles than the same stent deployed to 4 mm.

In some embodiments, the polygonal ICC 36 has six sides and has two inner reflex angles 72, two inner acute angles 70 and two inner angles 71 that may be acute, obtuse or right. Inner angles 71 may be about 90 degrees (plus of minus 15 degrees). Although some of the inner “corners” of the ICC polygon are rounded, such as with the inner portions of the peaks 26 and/or valleys 28, they are to be considered to be inner corners of a polygon.

As can be seen in FIG. 3, upon expansion of the stent 10, the segments 52, 54, of the struts 46, 48, 50, move in a direction of aligning 80 with the serpentine band 18 relative to their positions when the stent 10 is in an unexpanded state, such as in FIG. 2. When the stent is expanded and the ICC's 36 are expanded, as shown in FIG. 3, the first segment 52 of strut 46 and the second segment 54 of strut 50 are largely aligned with the serpentine band 18. In this, it is meant that they are at an angle 81 of less than 45° with the axis 80 of the serpentine band. Also, when the ICC's 36 are fully and the juncture 85 between segment 54 of strut 48 and segment 54 of strut 50 are substantially aligned with the axis 80 of the serpentine band 18.

Also, as can be seen in FIG. 3, segment 52 of strut 46 is substantially parallel with and distal to (relative to the length of the stent) segment 54 of strut 50. Segment 54 of strut 46 is substantially parallel with segment 52 of strut 50 and segments 52 and 54 of strut 48 are substantially parallel to one another. In some embodiments, these relative positions are substantially maintained from the ICC's 36 unexpanded state, as shown in FIG. 2, to their expanded state, as shown in FIG. 3.

In the embodiment shown in the figures, with regard to the linear portions of the segments, segment 52 of strut 46 and segment 54 of strut 50 are substantially the same length and are both shorter than the remaining segments of struts 46, 48 and 50. Segments 52 and 54 of strut 48 may be substantially the same length and segment 54 of strut 46 and segment 52 of strut 50 may be substantially the same length.

In some embodiments of the inventive stent, as shown in FIG. 3, segment 52 of strut 46 and segment 54 of strut 50 have widths that are less than that of their corresponding paired segments. The widths of both segments 52 and 54 of struts 46 and 50 may also be substantially uniform along their linear portions. The widths of segments 52 and 54 of strut 48 may have widths that inversely vary along their linear portion lengths. Segment 52 of strut 48 increases in width from its connection with segment 52 of strut 46 to its connection to segment 52 of strut 50 and segment 54 inversely decreases in width from its connection to segment 54 of strut 46 to its connection to segment 54 of strut 50. The invention also contemplates theses various thickness designs without noticeable hinge 60 thinning. The parameters may be designed to optimize the opening characteristics of the ICC structure and dependant upon the specifics of the particular stent design and ICC structure which is incorporated into the stent.

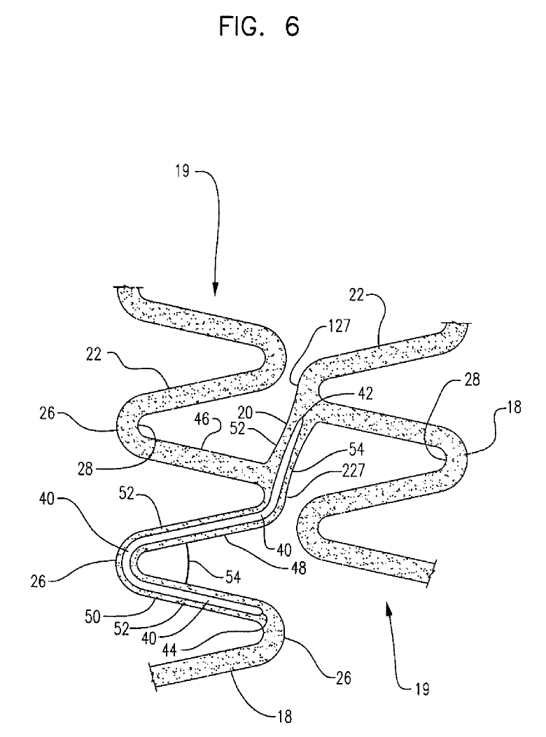

In some embodiments, as shown in FIG. 6, instead of the first end 42 of a slit 40 being located in the first strut 46, it 42 is located in a interconnecting strut 20. The interconnecting strut 20 is measured from the apex 127 of the peak 26 of the adjacent band 18 to which the interconnecting strut 20 is connected to the apex 227 of the peak 26 that is formed from the first strut 46 and the second strut 48. The length of the interconnecting strut 20 is defined by the distance between apex 127 and apex 227. In some embodiments, the first end 42 is located in the first half of the interconnecting strut 20 starting at apex 127. In some embodiments, the first end 42 is located in the first third of the interconnecting strut 20 starting at apex 127. The ending 44 positioning of the slit 40 may be as described above in reference to FIG. 4.

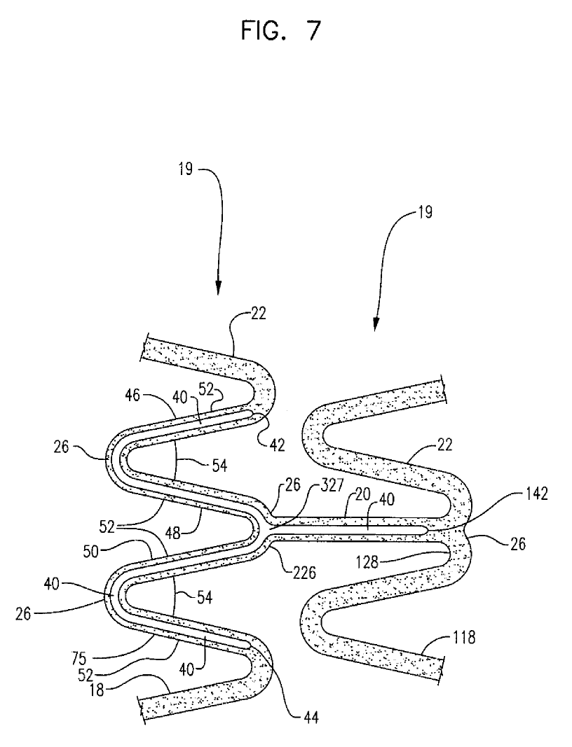

In some embodiments, as shown in FIG. 7, the slit 40, as shown and measured in FIG. 4, may extend into an interconnecting strut 20 to a third end 142. In this particular embodiment, the interconnecting strut 20 is a peak 226 to trough or valley 128 interconnecting strut, however it could be a peak to peak strut. Although the slit 40 may terminate 44 in the third strut 50, as shown in FIG. 4, in some embodiments, as shown in FIG. 7, it 40 may extend into a fourth shut 75. The location of the second end 44 in this case is positioned in the fourth strut 75 at the same place that it would be in the third strut 50, as described above in reference to FIG. 4.

In the embodiment shown in FIG. 7, the interconnecting strut 20 is measured from the valley 128 of the adjacent band 118 to which the interconnecting strut 20 is connected to the virtual apex 327 of the peak 26 that is formed from the second strut 48 and the third strut 50. It should be understood that in some embodiments that the interconnecting strut 20 may extend from the peak 26 that is formed from the first 46 and second 48 struts and that in some embodiments that the interconnecting strut 20 may extend from the peak 26 that is formed from the third 50 and fourth 75 struts. The length of the interconnecting strut 20 is defined by the distance between the valley 128 of the adjacent band 118 to the virtual apex 327. In some embodiments, the third end 142 is located in the first half of the interconnecting strut 20 starting at the valley 128. In some embodiments, the third end 142 is located in the first third of the interconnecting strut 20 starting at valley 128.

The present invention also contemplates stents having any, some or all of the slit 40 designs described herein.

In the above discussed embodiments, the inventive stents are of substantially uniform diameter. It is also within the scope of the invention to modify the stent patterns discussed above to prepare stents of non-constant diameter. For example, stent which taper in the expanded state may be made by decreasing the amplitude of the serpentine bands from one end of the stent to the other, or just along a desired portion of the stent. A tapered portion may be provided anywhere along the stent. For example, half of the stent, starting at one end of the stent, may be provided with a taper. Another way to achieve a tapered expanded stent is to change the stiffness of the serpentine bands and/or the connectors such that the stiffness of the serpentine bands and/or connectors varies along the length of the stent. The stiffness of the serpentine bands and/or connectors can be changed by altering length, width or thickness, adding additional stiffening material, using a chemical or mechanical means to alter the physical properties of the stent material, or applying one or a series of elastic elements about the stent.

The inventive stent patterns disclosed herein may also be used in conjunction with other known stent designs to provide stents whose properties vary over the length or portions thereof. The inventive slit patterns may also be used in non-serpentine stent designs, such as helix design, tri-bonate design, etc.

The invention is further directed to methods of manufacturing a stent according to the designs disclosed herein. The invention is further directed to methods of delivering and expanding a stent as described herein.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers Examples of suitable metals include, but are not limited to, stainless steel, titanium, tantalum, platinum, tungsten, gold and alloys of any of the above-mentioned metals Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials that are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The present invention may be incorporated into both of the two basic types of catheters used in combination with a guide wire, commonly referred to as over-the-wire (OTW) catheters and rapid-exchange (RX) catheters. The construction and use of both over-the-wire and rapid-exchange catheters are well known in the art.

In some embodiments, the stent, the delivery system of other portion of the assembly may include one or more areas, bands, coatings, members, etc. that is(are) detectable by imaging modalities such as X-Ray, MRI, ultrasound, etc. In some embodiments at least a portion of the stent and/or adjacent assembly is at least partially radiopaque.

In some embodiments, at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The inventive stents may further comprise a polymer coating in addition to or in place of the therapeutic coating. Suitable polymer coating materials include polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof, coatings from polymer dispersions such as polyurethane dispersions, for example, BAYHDROL RTM., fibrin, collagen and derivatives thereof, polysaccharides such as celluloses, starches, dextrans, alginates and derivatives, hyaluronic acid, squalene emulsions. Polyacrylic acid, available as HYDROPLUS RTM. (Boston Scientific Corporation, Natick, Mass.), and described in U.S. Pat. No. 5,091,205, the disclosure of which is hereby incorporated herein by reference, is particularly desirable. In a particular desirable embodiment of the invention, the polymer is a copolymer of polylactic acid and polycaprolactone.

In use, the stents disclosed herein are typically delivered via catheter to a desired bodily location. The choice of catheter will depend on the type of stent that is used and on the location to which the stent is delivered.

Any suitable method may be used to manufacture the inventive stents. For example, in addition to the methods listed above, the inventive stents may also be manufactured by preparing individual portions of the stent and connecting them to one another via welding, the use of adhesives or any other suitable joining technique. This list of manufacturing techniques is not meant to be exhaustive. Other manufacturing techniques may also be used to manufacture the inventive stents.

The invention will now be further described by the following numbered paragraphs:

1. A stent having a proximal end, a distal end, a longitudinal axis extending through the proximal and distal ends and an unexpanded state, wherein the stent is expandable from the unexpanded state to an expanded state, the stent further comprising:

a plurality of axially spaced bands, each band comprising a plurality of struts, wherein adjacent struts are connected to each other forming a plurality of peaks and troughs;

a plurality of interconnecting struts axially connecting the bands; and

a plurality of cells defined by axially adjacent bands and circumferentially adjacent interconnecting struts,

wherein, when the stent is in its unexpanded state, each band comprises a plurality of slits, each slit being non-linear and continuous from a first end to a second end and being formed in at least a portion of each of three consecutively connected struts.

2. The stent of paragraph 1, wherein the plurality of axially spaced bands are serpentine bands and, upon expansion of the stent to its expanded state, each of the slits expands in size to form an intra-columnar cell (ICC).

3. The stent of paragraph 2, each serpentine band having an axis circumferentially oriented around the longitudinal axis of the stent and a proximal side and a distal side bisected by the axis of the serpentine band, wherein the plurality of struts are spaced along the axis of the serpentine band and wherein the peaks have apex points and the troughs have center points, said peaks and troughs facing distally and proximally in an alternating manner.

4. The stent of paragraph 3, wherein the three consecutively connected struts comprise a first strut, a second strut and third strut, the first, second and third struts each having a first end and a second end, wherein the second end of the first strut is connected to the first end of the second strut and the second end of the second strut is connected to the first end of the third strut and wherein the first end of the slit is positioned in the first strut and the second end of the slit is positioned in the third strut.

5. The stent of paragraph 4, wherein the slit crosses the axis of the serpentine band at least three times.

6. The stent of paragraph 4, each of the first, second and third struts having a first segment and a second segment, wherein the slit separates the first segment and second segment of the each of the first, second and third struts.

7. The stent of paragraph 6, wherein the first segment of the first strut is connected to the first segment of the second strut forming a trough, the first segment of the second strut is connected to the first segment of the third strut forming a peak, the second segment of the first strut is connected to the second segment of the second strut forming a peak and the second segment of the second strut is connected to the second segment of the third strut forming a trough.

8. The stent of paragraph 3, wherein the ICC is substantially a polygon and has at least two inner reflex angles.

9. The stent of paragraph 8, wherein the ICC has at least two inner acute angles.

10. The stent of paragraph 9, wherein the ICC has at least few inner angles less than 180°.

11. The stent of paragraph 7, wherein the ICC is substantially a polygon and has at least two inner reflex angles.

12. The stent of paragraph 11, wherein the ICC has at least two inner acute angles.

13. The stent of paragraph 12, wherein the ICC has at least four inner angles less than 180°.

14. The stent of paragraph 11 wherein the first and second segments of the first, second and third struts define the ICC and wherein the first segment of the first strut is substantially parallel with the second segment of the third strut.

15. The stent of paragraph 11, wherein the first and second segments of the first, second and third struts define the ICC and wherein the second segment of the first strut is substantially parallel with the first segment of the third strut.

16. The stent of paragraph 11, wherein the first and second segments of the first, second and third struts define the ICC and wherein the first segment of the second strut is substantially parallel with the second segment of the second strut.

17. The stent of paragraph 14, wherein the second segment of the first strut is substantially parallel with the first segment of the third strut and wherein the first segment of the second strut is substantially parallel with the second segment of the second strut.

18. The stent of paragraph 7, wherein the second segment of the first strut and the first segment of the third strut have greater widths than the first segment of the first strut and the second segment of the third strut.

19. The stent of paragraph 18, wherein the first segment and second segment of the second strut vary in their width along their lengths.

20. The stent of paragraph 11, the serpentine bands further comprising a plurality of primary hinge points, wherein, upon expansion of the stent and the forming of the ICC, the first and second segments of the first, second and third struts rotate around the primary hinge points, increasing the size of the slit to form the ICC.

21. The stent of paragraph 20, a first primary hinge point being located in an end of the first segment of the second strut, wherein the first segment of the second strut and the first segment of the third strut pivot around the first primary hinge point.

22. The stent of paragraph 21, a second primary hinge point being located in an end of the second segment of the second strut, wherein the second segment of the second strut and the second segment of the first strut pivot around the second primary hinge point.

23. The stent of paragraph 22, wherein the ICC is defined by the first and second segments of the first, second and third struts, which have six primary hinge points.

24. The stent of paragraph 3, wherein the shapes of the plurality of cells are different that the shape of the ICC.

25. The stent of paragraph 3, wherein the stent further comprises a therapeutic agent.

26. The stent of paragraph 25, wherein the therapeutic agent is in the form of a coating or layer on the outer surface of the stent.

27. The stent of paragraph 26, wherein the therapeutic agent is chosen from the group consisting of non-genetic therapeutic agents, genetic materials, cells and combinations thereof.

28. A stent delivery system comprising a catheter having a distal portion and a stent according to paragraph 1.

29. A method of delivering a stent to a site comprising the steps of:
providing a stent delivery system, the stent delivery system comprising:
   a stent delivery catheter, and
   a stent according to paragraph 1,
advancing the stent delivery system to a vessels site; and
deploying the stent at the vessel site.

30. A stent having a proximal end, a distal end, a longitudinal axis extending through the proximal and distal ends and an unexpanded state, wherein the stent is expandable from the unexpanded state to an expanded state, the stent further comprising:

a plurality of axially spaced bands, each band comprising a plurality of struts, wherein adjacent struts are connected to each other forming a plurality of peaks and troughs;

a plurality of interconnecting struts axially connecting the bands; and

a plurality of cells defined by axially adjacent bands and circumferentially adjacent interconnecting struts,

wherein, when the stent is in its unexpanded state, each band comprises a plurality of slits, each slit being non-linear and continuous from a first end, which is located within one of the interconnecting struts, to a second end and being formed in at least a portion of each of two consecutively connected struts.

31. The stent of paragraph 30, wherein the plurality of axially spaced bands are serpentine bands and, upon expansion of the stent to its expanded state, each of the slits expands in size to form an intra-columnar cell (ICC).

32. A stent having a proximal end, a distal end, a longitudinal axis extending through the proximal and distal ends and an unexpanded state, wherein the stent is expandable from the unexpanded state to an expanded state, the stent further comprising:

a plurality of axially spaced bands, each band comprising a plurality of struts, wherein adjacent struts are connected to each other forming a plurality of peaks and troughs;

a plurality of interconnecting struts axially connecting the bands; and

a plurality of cells defined by axially adjacent bands and circumferentially adjacent interconnecting struts,

wherein, when the stent is in its unexpanded state, each band comprises a plurality of slits, each slit being non-linear and continuous from a first end to a second end and being formed in at least a portion of each of three consecutively connected struts and wherein each slit extends into an adjacent interconnecting strut.

33. The stent of paragraph 32, wherein the second end of each slit is located within a fourth strut, the fourth strut being consecutively connected to the three consecutively connected struts.

34. The stent of paragraph 32, wherein the plurality of axially spaced bands are serpentine bands and, upon expansion of the stent to its expanded state, each of the slits expands in size to from an intra-columnar cell (ICC).

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

The particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below (e.g. claim 4 may be taken as alternatively dependent on claim 2, or on claim 3; claim 5 may be taken as alternatively dependent from any of claims 1-3, etc.).

This completes the description of the preferred and alternate embodiments of the invention. Those skilled in the art may recognize other equivalents to the specific embodiment described herein which equivalents are intended to be encompassed by the claims attached hereto.

1. A stent having a proximal end, a distal end, a longitudinal axis extending through the proximal and distal ends and an unexpanded state, wherein the stent is expandable from the unexpanded state to an expanded state, the stent further comprising:
a plurality of axially spaced bands, each band comprising a plurality of struts, wherein adjacent struts are connected to each other forming a plurality of peaks and troughs;
a plurality of interconnecting struts axially connecting the bands; and
a plurality of cells defined by axially adjacent bands and circumferentially adjacent interconnecting struts,
wherein, when the stent is in its unexpanded state, each band comprises a plurality of slits, each slit being non-linear and continuous from a first end to a second end and being formed in at least a portion of each of three consecutively connected struts.

2. The stent of claim 1, wherein the plurality of axially spaced bands are serpentine bands and, upon expansion of the stent to its expanded state, each of the slits expands in size to form an intra-columnar cell (ICC).

3. The stent of claim 2, each serpentine band having an axis circumferentially oriented around the longitudinal axis of the stent and a proximal side and a distal side bisected by the axis of the serpentine band, wherein the plurality of struts are spaced along the axis of the serpentine band and wherein the peaks have apex points and the troughs have center points, said peaks and troughs facing distally and proximally in an alternating manner.

4. The stent of claim 3, wherein the three consecutively connected struts comprise a first strut, a second strut and third strut, the first, second and third struts each having a first end and a second end, wherein the second end of the first strut is connected to the first end of the second strut and the second end of the second strut is connected to the first end of the third strut and wherein the first end of the slit is positioned in the first strut and the second end of the slit is positioned in the third strut.

5. The stent of claim 4, wherein the slit crosses the axis of the serpentine band at least three times.

6. The stent of claim 4, each of the first, second and third struts having a first segment and a second segment, wherein the slit separates the first segment and second segment of the each of the first, second and third struts.

7. The stent of claim 6, wherein the first segment of the first strut is connected to the first segment of the second strut forming a trough, the first segment of the second strut is connected to the first segment of the third strut forming a peak, the second segment of the first strut is connected to the second segment of the second strut forming a peak and the second segment of the second strut is connected to the second segment of the third strut forming a trough.

8. The stent of claim 3, wherein the ICC is substantially a polygon and has at least two inner reflex angles.

9. The stent of claim 8, wherein the ICC has at least two inner acute angles.

10. The stent of claim 9, wherein the ICC has at least four inner angles less than 180°.

11. The stent of claim 7, wherein the second segment of the first strut and the first segment of the third strut have greater widths than the first segment of the first strut and the second segment of the third strut.

12. The stent of claim 3, wherein the shapes of the plurality of cells are different that the shape of the ICC.

13. The stent of claim 3, wherein the stent further comprises a therapeutic agent.

14. The stent of claim 13, wherein the therapeutic agent is in the form of a coating or layer on the outer surface of the stent.

15. The stent of claim 14, wherein the therapeutic agent is chosen from the group consisting of non-genetic therapeutic agents, genetic materials, cells and combinations thereof.

16. A stent having a proximal end, a distal end, a longitudinal axis extending through the proximal and distal ends and an unexpanded state, wherein the stent is expandable from the unexpanded state to an expanded state, the stent further comprising:
a plurality of axially spaced bands, each band comprising a plurality of struts, wherein adjacent struts are connected to each other forming a plurality of peaks and troughs;
a plurality of interconnecting struts axially connecting the bands; and
a plurality of cells defined by axially adjacent bands and circumferentially adjacent interconnecting struts,
wherein, when the stent is in its unexpanded state, each band comprises a plurality of slits, each slit being non-linear and continuous from a first end, which is located within one of the interconnecting struts, to a second end and being formed in at least a portion of each of two consecutively connected struts.

17. The stent of claim 16, wherein the plurality of axially spaced bands are serpentine bands and, upon expansion of the stent to its expanded state, each of the slits expands in size to form an intra-columnar cell (ICC).

18. A stent having a proximal end, a distal end, a longitudinal axis extending through the proximal and distal ends and an unexpanded state, wherein the stent is expandable from the unexpanded state to an expanded state, the stent further comprising:
a plurality of axially spaced bands, each band comprising a plurality of struts, wherein adjacent struts are connected to each other forming a plurality of peaks and troughs;
a plurality of interconnecting struts axially connecting the bands; and
a plurality of cells defined by axially adjacent bands and circumferentially adjacent interconnecting struts,
wherein, when the stent is in its unexpanded state, each band comprises a plurality of slits, each slit being non-linear and continuous from a first end to a second end and being formed in at least a portion of each of three consecutively connected struts and wherein each slit extends into an adjacent interconnecting strut.

19. The stent of claim 18, wherein the second end of each slit is located within a fourth strut, the fourth strut being consecutively connected to the three consecutively connected struts.

20. The stent of claim 18, wherein the plurality of axially spaced bands are serpentine bands and, upon expansion of the stent to its expanded state, each of the slits expands in size to form an intra-columnar cell (ICC).
